(19) United States
(12) Patent Application Publication
(10) Pub. No.: US 2005/0202111 A1
(43) Pub. Date: Sep. 15, 2005

(54) Anti-Parkinson's disease pharmaceutical and method of use

(76) Inventors: Bala V. Manyam, Temple, TX (US); Muralikrishnan Dhanasekaran, Temple, TX (US); John M. Cassady, Columbus, OH (US)

Correspondence Address: SIDLEY AUSTIN BROWN & WOOD LLP;ATTN: DC PATENT DOCKETING, 1501 K STREET, N.W., WASHINGTON, DC, 20005, US

(21) Appl. No.: 11/047,760

(22) Filed: Feb. 2, 2005

Related U.S. Application Data
(60) Provisional application No. 60/549,999, filed on Mar. 4, 2004.
(60) Provisional application No. 60/569,786, filed on May 10, 2004.

(51) Int. Cl. A61K 35/78

(52) U.S. Cl. 424/774

(57) ABSTRACT

Employing a novel method of extracting Mucuna pruriens cotyledons, a composition for the treatment of Parkinson's disease has now been found which provides effective immediate relief of symptoms and also slows down the disease process. Because this novel composition can be given to patients by multiple routes of administration including injection, the Mucuna pruriens cotyledon extract of the present invention provides a distinct advantage over known Mucuna pruriens powder and extract preparations.

### TECHNICAL FIELD OF INVENTION

This application relates to methods of making drugs or bio-affecting compositions and method of use. In particular, this invention relates to preparation of an anti-Parkinson's disease composition and therapeutic use thereof.

### BACKGROUND OF THE INVENTION

Parkinson's disease (i.e., paralysis agitans or shaking palsy) is a degenerative disease of the nervous system associated with the depletion of or interference with the neurotransmitter dopamine within the basal ganglia. The disease is characterized by hypokinetic movement disorders including resting tremors, muscular rigidity, poverty of movement; difficulty with balance and walking, depression, and dementia. With today's increased life expectancy comes an increase in the number of people at risk for developing Parkinson's disease.

To date, pharmacological treatment for Parkinson's disease has focused on alleviating symptoms or the deleterious side effects of prescribed drugs. One major focus of Parkinson's disease research is the development of neuroprotective drugs that slow or halt the disease progression by preventing or blocking the behavioral or biochemical toxic insults induced by neurotoxins, and the development of neurorestorative drugs that have the ability to regenerate biochemical activity and recover physiological function(s) after toxic insult induced by neurotoxin.

Current pharmacological treatment for Parkinson's disease generally includes one or more of the following synthetic drugs: dopaminergic agents such as levodopa (also referred to as L-DOPA; L-3,4-dihydroxyphenylalanine), a metabolic precursor of dopamine (3,4-dihydroxyphenylethylamine), either singly or in combination with decarboxylase inhibitors such as carbidopa and benserazide; dopamine agonists such as bromocriptine, pramipexole, ropinirole or pergolide; monoamine oxidase-B (MAO_{B}) inhibitor such as selegiline; anticholinergics such as trihexyphenidyl, benztropine mesylate, procyclidine, biperiden, and ethopropazine; antihistamines such as diphenhydramine and dorphenadrine; and amantadine. Depression is treated symptomatically with suitable antidepressants.

Most symptoms are reportedly controlled through use of these various synthetic drugs; however, no single drug has been found to alleviate all symptoms or to actually affect the progression of disease. While currently available synthetic antiparkinson drugs like levodopa and dopamine agonists benefit motor symptoms, they have no proven neuroprotective benefit. Adverse effects such as dyskinesia have also been reported with long-term levodopa treatment (Martignoni, et al. 2003. “Motor complications of Parkinson's disease,” Neurol Sci Suppl 1:S27-29), highlighting the need for further antiparkinson drug development.

Coenzyme Q-10 (ubiquinone, 2-methyl-5,6-dimethoxy-1,4-benzoquinone) is a biologically occurring fat soluble quinone considered vital to the optimal functioning of an organism. The primary role of coenzyme Q-10 is to transfer electrons between redox components of the electron transport chain to create a proton gradient across the inner mitochondrial membrane, thereby driving ATP formation. Additional functions of coenzyme Q-10 include influencing membrane fluidity, recycling radical forms of vitamin C and E, and most importantly acting as a lipid antioxidant protecting membrane phospholipids against peroxidation. The antioxidant and neuroprotective activity of coenzyme Q-10 was demonstrated in animal models where coenzyme Q-10 administered orally protected the nigrostriatal dopaminergic system in one-year-old mice treated with MPTP, a toxin detrimental to the nigrostriatal dopaminergic system (Beal, et al. 1998. “Coenzyme Q-10 attenuates the 1-methyl-4-phenyl-1,2,3,6 tetrahydropyridine (MPTP) induced loss of striatal dopamine and dopaminergic axons in aged mice,” Brain Res 783:109-114). Coenzyme Q-10 also reportedly protects against striatal lesions produced by both malonate and 3-nitropropionic acid (Matthews, et al. 1998. “Coenzyme Q10 administration increases brain mitochondrial concentrations and exerts neuroprotective effects,” Proc Natl Acad Sci USA 95:8892-8897). These studies showed that coenzyme Q-10 plays a significant role in cellular dysfunction found in Parkinson's disease and is a potential antioxidant and neuroprotective drug for parkinsonian patients. Numerous studies have confirmed that patients with Parkinson's disease have reduced complex-I activity of the electron transport chain in brain and platelets. Platelet mitochondria from parkinsonian patients were found to have lower levels of coenzyme Q-10 than mitochondria from age/sex-matched controls (Gotz, et al. 2000. “Altered redox state of platelet coenzyme Q10 in Parkinson's disease,” J Neural Transm 107:41-48). There are also clinical reports showing oral synthetic coenzyme Q-10 was well absorbed in patients suffering from Parkinson disease and caused a trend toward increased complex-I activity (Shults, et al. 1997. “Coenzyme Q10 levels correlate with the activities of complexes I and II/III in mitochondria from parkinsonian and non-parkinsonian subjects,” Ann Neurol 42:261-264). Coenzyme Q-10 has also been reported to offer neuroprotective benefit, especially if used at high doses (1200 milligrams/day) (Shults, et al. 2002. “Effects of coenzyme Q10 in early Parkinson disease: evidence of slowing of the functional decline,” Arch Neurol 59:1541-1550; Hunter, D A. 2003. “Coenzyme Q10 in early Parkinson disease,” Arch Neurol 60:1170-1172). However, coenzyme Q-10 is not known to provide immediate strong symptomatic benefit on tremor, rigidity, poverty of movement and other disabling motor concerns of patients with Parkinson's disease (Muller, et al. 2003. “Coenzyme Q10 supplementation provides mild symptomatic benefit in patients with Parkinson's disease,” Neurosci Lett 341:201-204).

Based on the fact that nicotinamide adenine dinucleotide (NADH) stimulates tyrosine hydroxylase and dopamine biosynthesis in human brain and tissue culture, NADH has been used in Parkinson's disease treatment. In an open clinical trial, synthetic NADH was parenterally administered to patients with Parkinson's disease (Birkmayer G J and Birkmayer W. 1989. “Stimulation of endogenous L-dopa biosynthesis—a new principle for the therapy of Parkinson's disease. The clinical effect of nicotinamide adenine dinucleotide (NADH) and nicotinamide adenine dinucleotidephosphate (NADPH),” Acta Neurol Scand 126:183-1877; Birkmayer et al. 1989. “The coenzyme nicotinamide adenine dinucleotide (NADH) improves the disability of parkinsonian patients,” J Neural Transm Park Dis Dement Sect 1:297-302; and Birkmayer W and Birkmayer G J. 1989. “Nicotinamid-adenindinucleotide (NADH): the new approach in the therapy of Parkinson's disease,” Ann Clin Lab Sci 19: 38-43). In all patients, a beneficial clinical effect was observed, with the best results obtained with a dose of 25 to 50 mg every second day by intravenous administration. Concomitantly, with the improvement in disability, the urine homovanillic acid (a metabolite of dopamine) level increased significantly, indicating a stimulation of endogenous levodopa biosynthesis (Birkmayer et al. 1990. “The clinical benefit of NADH as stimulator of endogenous L-dopa biosynthesis in parkinsonian patients,” Adv Neurol 53:545-549). The orally applied form of NADH also yielded an overall improvement and was comparable to that of the parenterally applied form (Birkmayer et al. 1993. “Nicotinamide adenine dinucleotide (NADH)—a new therapeutic approach to Parkinson's disease. Comparison of oral and parenteral application.” Acta Neurol Scand 146: 32-35).

Polyphenols have been shown to significantly inhibit hydrogen peroxide-induced lipid peroxidation, suggesting its use in the treatment of Parkinson's disease. A number of studies have shown that polyphenols can block neuronal death in vitro, and may have therapeutic properties in animal models of neurodegenerative diseases including Parkinson's disease (Di Matteo V and Esposito E. 2003. “Biochemical and therapeutic effects of antioxidants in the treatment of Alzheimer's disease, Parkinson's disease, and amyotrophic lateral sclerosis.” Curr Drug Target CNS Neurol Disord 2: 95-107). Polyphenols have the ability to cross the blood-brain barrier and can exert their antioxidant and iron-chelating properties in the brain, which suggest polyphenol compounds, may be an important class of drugs to be developed for treatment of neurodegenerative diseases where oxidative stress has been implicated. Polyphenols such as (−)-epigallocatechin-3-gallate prevents N-methyl-4-phenyl-1,2,3,6-tetrahydropyridine induced dopaminergic neurodegeneration resulting in parkinsonism that is used as an animal model of Parkinson's disease (Levites, et al. 2001. “Green tea polyphenol (−)-epigallocatechin-3-gallate prevents N-methyl-4-phenyl-1,2,3,6-tetrahydropyridine-induced dopaminergic neurodegeneration,” J Neurochem 78: 1073-1082).

The major antioxidant molecules in the brain are glutathione, superoxide dismutase and catalase. Glutathione is a tripeptide containing glutamic acid, cysteine and glycine, and serves as a coenzyme in a variety of enzyme reactions. It primarily acts by reducing inactive disulfide linkages of enzymes to active sulfhydryl groups, while the sulfhydryl groups of glutathione get oxidized. Mitochondrial glutathione play an important role in the defense against endogenous membrane peroxidation and subsequent changes by reducing H₂O₂ via glutathione-peroxidase. The levels of glutathione are believed to be rate-limiting step in the process of detoxification of H₂O₂ or other peroxides. In humans, glutathione is present in blood, liver and brain. It has been also suggested that thiol groups are essential for complex-I activity and other respiratory mitochondrial enzymes. In idiopathic Parkinson's disease, glutathione is depleted in the nigrostriatal tract (Perry, et al. 1982. “Parkinson's disease: A disorder due to nigral glutathione deficiency?” Neurosci Lett 33:305-310). In normal physiology, the brain is subjected to oxidative insult due to its high metabolic turnover such as a consequence of its high content of phospholipids, unsaturated fatty acids, high consumption of oxygen, increased generation of superoxide anion as well as due to its relatively low levels of antioxidant molecules. During this normal metabolism the neurons and the supporting cells generate reactive oxygen species such as superoxide anions, hydroxyl radicals, singlet oxygen, and H₂O₂. Recent experiments showed that the thiol antioxidant N-acetylcysteine (NAC) can protect against age-related decrease in complex-I activity (Martinez Banaclocha M. 2000. “N-acetylcysteine elicited increase in complex I activity in synaptic mitochondria from aged mice: implications for treatment of Parkinson's disease,” Brain Res 859: 173-175). The sulfhydryl group can facilitate the redox reaction and is considered to play an important role in scavenging toxic reactive oxygen species. The sulfhydryl group is present in naturally occurring glutathione and in N-acetylcysteine which are discussed above.

Drugs derived from plant sources have also been reported to be effective in the treatment of Parkinson's disease. In particular, the seeds of Mucuna pruriens and other members of the Mucuna species were described as useful therapeutic agents in various diseases of the human nervous system including Parkinson's disease in the ancient Indian medical system Ayurveda. The genus Mucuna, belonging to the family Leguminosae, includes Mucuna pruriens, Mucuna andrena, Mucuna birdwoodiana, Mucuna deeringiana, Mucuna holtini, Mucuna irukande, Mucuna mutisiana, Mucuna sempervirens, and Mucuna slonei. Mucuna pruriens is a twiner with trifoliate leaves, purple flowers, and turgid S-shaped pods covered with hairs and represents the most cultivated and investigated species.

Mucuna pruriens seeds are well known to contain levodopa (Bell EA and Janzen D H. 1971. “Medical and ecological considerations of L-dopa and 5-HTP in seeds,” Nature 229:136-137; and Damodaran M. and Ramaswamy R. 1937. “Isolation of L-dopa from the seeds of Mucuna pruriens,” Biochemistry 31:2149-2151). In addition to levodopa, Mucuna pruriens seeds are reported to contain several known compounds including N,N-diemethyltryptamine; 5-methoxy-N,N-dimethytryptamine; N,N-dimethyltryptamine N-oxide; bufotenine; choline; serotonin; 2-amino-(3,4-dihydroxyphenyl)propanic acid; 6-hydroxy-1-methyl-beta-carboline; mucuadine; mucuadinine; mucuadininine; mucunadine; mucnine; prurienidine; prurienine; prurieninine; 5-hydroxytryptamine; and 1,2,3,4-tetrahydro-6,7-dihydroxy-3-isoquinolinecarboxylic acid, and other unidentified compounds.

In 1978, Vaidya et al reported a study of patients with Parkinson's disease who were initially treated with synthetic levodopa followed by treatment with a powder made from the whole bean of Mucuna, wherein the patients experienced a decrease in the incidence of adverse effects during treatment with the Mucuna powder compared to the synthetic levodopa treatment (Vaidya, et al. 1978. “Treatment of Parkinson's disease with the cowhage plant—Mucuna pruriens bak,” Neurol India 26:171-176). Approximately 40 to 60 grams of the whole Mucuna bean powder were administered in four divided doses per day, and although tolerated at this dose, the patients reported undesirable taste and bulkiness of the dose.

U.S. Pat. No. 6,106,839 discloses a composition for the treatment of Parkinson's disease comprising 55-99% Mucuna pruriens, 10-35% Piper longum, and 5-15% Zingiber officinalis. Each of the botanicals is prepared in powder form and mixed together, and the mixture is used in the manufacture of capsules, tablets, syrups for administration to patients.

India Patent No. 174,540 discloses a water-soluble formulation for the treatment of Parkinson's disease comprising pulverized ripened dry Mucuna pruriens seeds in powder form, an antioxidant such as Vitamin C or Vitamin E, a stabilizer such as gum polysaccharine, and optionally, flavor, fragrances and a taste enhancer. Also disclosed is a process for preparing the plant-based Ayurvedic formulations comprising the steps of selecting the active ingredient from Mucuna pruriens bak plant (ripened seeds), pulverizing the active ingredient into a fine powder, and mixing antioxidant and stabilizer with the powder. The powder is then mixed with water and administered orally.

U.S. Pat. No. 3,253,023 discloses a method for isolating levodopa (L-DOPA) from ground velvet beans such as Stizolobium deeringianum (Stizolobium is considered a subgenus of Mucuna which includes velvet beans). Levodopa was recovered by (1) extracting ground velvet beans in a dilute 1-10% organic acid solution consisting of formic, acetic, chloroacetic and propionic acids at 10-80 degrees C. for about 10-20 hours; (2) separating the extraction liquid from the bean pulp by decantation and filtration through acid-washed diatomaceous earth; (3) concentrating the decanted liquid to 10-20% of its volume and treating with a sorbant such as acid-washed activated carbon; and (4) recovering levodopa therefrom.

Daxenbichler et al reported a levodopa (L-DOPA) recovery method from Mucuna seed, wherein (1) whole ground seed was repeatedly extracted in hot water; (2) the combined supernatant was passed through an ion exchange column; (3) the materials retained by the column were eluted with 10% acetic acid solution; (4) the eluate was concentrated; and (5) levodopa was crystallized by refrigeration overnight. The amount of levodopa recovered from eight Mucuna species ranged from 3.1-6.1%. (Daxenbichler, et al. 1972. “L-DOPA recovery from Mucuna seed,” J Agr Food Chem 20:1046-1048).

Tripathi and Upadhyay reported in vitro and in vivo studies to determine the effect of an alcohol extract of whole Mucuna pruriens seeds on free radicals and oxidative stress in albino rats. The alcohol extract was prepared by extracting ground whole seed in ethanol; distilled and concentrated under reduced pressure in a Buchi type rotary evaporator; vacuum desiccated to dry powder (yield, 40.2% w/w) (Tripathi, et al. 1996. “Studies on the inhibitory effect of S. nux vomica alcoholic extract on iron induced lipid peroxidation,” Phytomedicine III 20:175-180); and for use in biological experiments, the powder was suspended in a drug vehicle of Tween 80:water (1:9) at known concentrations (w/v). Albino rats of inbred Charles Foster strain (100-150 g body weight) were used in the in vitro and in vivo studies. The in vitro effect in rat liver homogenate was determined for the seed extract on the iron sulfate induced lipid peroxidation (ED₅₀=100 μg/ml), glutathione content (no protection), superoxide anion generation (ED₅₀=185 μg/ml), and hydroxylation of sodium salicylates (ED₅₀=0.5 mg/ml). In an in vivo study, the alcohol extract suspension was given orally to rats for 30 days, and measurements of iron sulfate induced lipid peroxidation; glutathione content; superoxide dismutase activity; and serum GOT, GPT, and SALP levels in liver homogenates obtained from the treated rats did not show any change from control rats, indicating a lack of toxicity up to 600 mg/kg body weight.

HP-200 is a commercially available palatable formulation comprising Mucuna pruriens cotyledon powder, a flavoring agent and a sweetener. Several studies in mammals have reported HP-200 as a safe and effective treatment for Parkinson's disease (Manyam, et al. 1995. “An alternative medicine treatment for Parkinson's disease: results of a multicenter clinical trial. HP-200 in Parkinson's disease study group,” J Altern and Complement Med 1:249-255; Mahajani, et al. 1996. “Bioavailability of L-DOPA from HP-200—a formulation of seed powder of Mucuna pruriens (Bak): a pharmacokinetic and pharmacodynamic study,” Phytotherapy Res 10:254-256; Hussain, G and Manyam, B V. 1997. “Mucuna pruriens proves more effective than L-DOPA in Parkinson's disease animal model,” Phytotherapy Res 11:419-423; and Manyam B V and Parikh K M. 2002. “Anti-Parkinson activity of Mucuna pruriens seeds,” Ann Neurosci 9:40-46). In a clinical trial with 60 patients, HP-200 was shown to be effective in controlling the symptoms of Parkinson's disease at a significant level (p<0.0001; t-test) (Manyam, et al. 1995. “An alternative medicine treatment for Parkinson's disease: results of a multicenter clinical trial. HP-200 in Parkinson's disease study group,” J Altern and Complement Med 1:249-255). The cotyledon powder was also shown to be 2-3 times more effective than the equivalent amount of synthetic levodopa in the 6-hydroxydopamine lesioned rat model of Parkinson's disease, indicating that the non-levodopa components present in the cotyledon powder either enhanced the activity of levodopa or have independent antiparkinsonian activity (Hussain, G and Manyam, B V. 1997. Phytotherapy Res 11:419-423; and Manyam B V and Parikh K M. 2002. Ann Neurosci 9:40-46). In the monkey model of Parkinson's disease, Mucuna pruriens cotyledon powder did not produce dyskinesia, a particularly troubling adverse effect associated with long-term antiparkinson levodopa therapy (Subramanian et al. 32^{nd} Annual Meeting for Society for Neurosciences, Nov. 2-7, 2002; abstract No. 787.4). Despite its antiparkinson activity, treatment with Mucuna pruriens cotyledon powder caused some deleterious side effects. Because the effective dose of the Mucuna pruriens cotyledon powder was “bulky” (2.5 and 5.0 g/kg in experimental animals; 15 gram/dose in humans), only oral administration was possible, and the effective therapeutic dose of the powder was not amenable to formulate into a capsule or tablet form. In studies with human subjects, the 15-gram doses given three times a day reportedly resulted in some degree of gastric intolerance (“bean effect”).

Employing a novel method of extracting Mucuna pruriens cotyledons, a composition for the treatment of Parkinson's disease has now been found which provides effective immediate relief of symptoms and also slows down the disease process. Because this novel composition can be given to patients by multiple routes of administration including injection, the Mucuna pruriens cotyledon extract of the present invention provides a distinct advantage over known Mucuna pruriens powder and extract preparations.

Each of the above referenced patents, applications, published applications and other publications are incorporated herein by reference in their entirety as if each such publication or document was specifically and individually indicated to be incorporated herein by reference. Citation of the above publications and documents is not intended as an admission that they are prior art, nor does it constitute any admission as to the contents or date of these publications or documents.

### SUMMARY OF THE INVENTION

In one aspect, the invention is a method for preparing an extract of Mucuna pruriens cotyledon powder comprising the steps of defatting the powder to form defatted solids, subjecting the defatted solids to an ethanolic extraction, and isolating and purifying the ethanolic extract. The extract is preferably freeze dried for increased shelf life.

In another aspect, the invention is an extract of Mucuna pruriens cotyledon powder made according to the extraction method of the present invention.

In another aspect, the invention is a therapeutic composition comprising an extract of Mucuna pruriens cotyledon powder made according to the extraction method of the present invention.

In another aspect, the invention is a method of treatment for Parkinson's disease or related disorder comprising administering an effective therapeutic amount of the extract of Mucuna pruriens cotyledon powder made according to the extraction method of the present invention.

### BRIEF DESCRIPTION OF THE DRAWINGS

FIG. 1 depicts the process steps for preparing for the Mucuna pruriens extract (MPX).

FIG. 2 depicts the efficacy of MPX in terms of dose-dependent contralateral rotations in the 6-OHDA treated rats (n=6) at varying MPX concentrations.

FIG. 3 depicts the efficacy of MPX in terms of dose-dependent scavenging activity on diphenylpicrylhydrazyl (DPPH) radicals (n=4). The scavenging activity of levodopa and ground Mucuna pruriens cotyledon powder (MPCP) on DPPH radicals was also measured for comparison. Values represent percentage inhibition, mean±SEM. “*” represents p<0.001.

FIG. 4 depicts the efficacy of MPX in terms of dose-dependent scavenging activity on 2,2′-azinobis(3-ethylbenzothiasoline-6-sulfonate) (ABTS) (n=4). The scavenging activity of levodopa was also measured for comparison. Values represent percentage inhibition, mean±SEM. “*” represents p<0.001.

FIG. 5 depicts the efficacy of MPX in terms of scavenging activity on hydroxyl radical adducts 2,3- and 2,5-dihydroxybenzoic acid (DHBA) as generated by the Fenton reaction (n=4). Values represent pmole of dihydroxy benzoic acid, mean±SEM. “*” represents p<0.001.

FIG. 6 depicts the efficacy of MPX in terms of dose-dependent inhibition of hydrogen peroxide induced lipid peroxidation (n=6). Data represents mean±SEM. “*” represents p<0.001.

FIG. 7 depicts the total in vitro antioxidant activity of MPX as measured by a commercial kit (Total antioxidant kit BIOXYTECH AOP-490™, OXIS International, Inc., Portland, Oreg.). The assay is based upon the reduction of divalent metal Cu⁺⁺ to monovalent Cu⁺ by the combined action of all the antioxidants present in the sample. A chromogenic reagent, Bathocuproine (2,9-dimethyl-4,7-diphenyl-1,10-phenanthroline), selectively forms a 2:1 complex with Cu+which has a maximum absorbance at 490 nm. A standard dilution series of water soluble antioxidant, uric acid, at known concentrations was used to create a calibration curve. The results refer to the mM uric acid equivalents of antioxidant activity (n=6). Data represents mean±SEM.

FIG. 8 depicts the effect of MPX on mitochondrial complex-I activity (n=6). The effect of Levodopa was also measured for comparison. Complex-I activity is represented as NADH oxidized/mg protein/min. Data represents mean±SEM. “*” represents p<0.05.

FIG. 9 depicts the fingerprint of MPX. MPX had 39 peaks as detected by HPLC-UV detector at 225 nm. The highest peak was detected around 8.8 minutes with a maximum deflection of 200-250 mvolt (Peaks detected at 2.825; 3.042; 4.983; 6.758; 7.367; 8.875; 10.000; 10.975; 11.875; 13.108; 14.850; 16.692; 18.333; 18.942; 20.167; 21.675; 24.958; 28.858; 29.683; 31.167; 32.067; 34.475; 36.000; 37.192; 38.692; 39.642; 42.027; 42.658; 46.593; 46.733; 48.017; 52.192; 52.817; 55.350; 57.908; 60.442; 64.408; 64.542; and 71.058 minutes).

FIG. 10 depicts the fingerprint HPLC of a Bacopa monniera extract. The Bacopa monniera extract had 65 peaks as detected by HPLC-UV detector at 225 nm. The highest peak was detected around 2.8 minutes with a maximum deflection of 400-500 mvolt (Peaks detected at 1.333; 2.008; 2.842; 3.017; 3.675; 4.025; 4.317; 5.042; 5.375; 5.683; 6.042; 6.875; 7.433; 8.125; 8.775; 9.517; 10.175; 10.550; 10.858; 11.650; 12.267; 12.983; 13.817; 14.958; 15.658; 16.083; 16.758; 18.117; 18.900; 20.858; 21.725; 22.425; 22.950; 23.575; 24.233; 24.775; 25.392; 25.975; 26.617; 28.100; 29.692; 32.808; 34.167; 37.400; 39.717; 41.192; 43.783; 44.317; 46.017; 48.767; 49.350; 49.817; 51.392; 51.958; 52.525; 53.617; 54.167; 54.758; 55.133; 72.383; 76.100; 76.917; 77.425; 78.742 and 79.267 minutes).

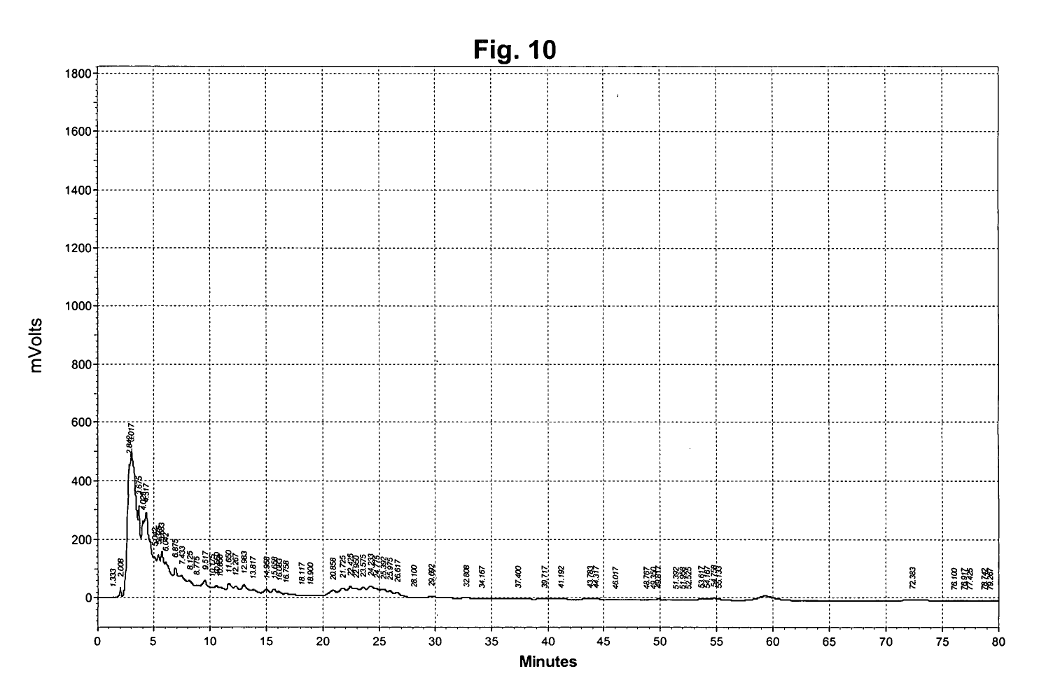

FIG. 11 depicts the HPLC fingerprint of a Centella asiatica extract. Centella asiatica extract had 75 peaks as detected by HPLC-UV detector at 225 nm. The highest peak was detected around 3.1 minutes with a maximum deflection of 1600-1700 mvolt (peaks detected at 2.650; 3.117; 3.517; 4.000; 4.242; 4.575; 5.325; 5.600; 5.950; 6.783; 7.158; 7.950; 8.825; 9.967; 10.625; 11.342; 11.767; 12.692; 13.067; 13.600; 14.608; 16.467; 17.875; 18.750; 19.292; 20.083; 20.817; 22.633; 24.717; 25.783; 27.208; 28.458; 29.700; 30.075; 30.675; 31.317; 32.583; 33.842; 34.450; 35.133; 35.508; 36.017; 37.242; 40.117; 41.308; 42.350; 45.500; 46.042; 46.617; 47.617; 49.708; 50.267; 50.733; 51.275; 51.983; 53.458; 54.025; 54.592; 55.042; 55.650; 57.425; 59.192; 59.792; 60.350; 60.975; 62.975; 63.575; 67.300; 67.817; 72.508; 73.083; 77.275; 78.317; 79.025 and 79.442 minutes).

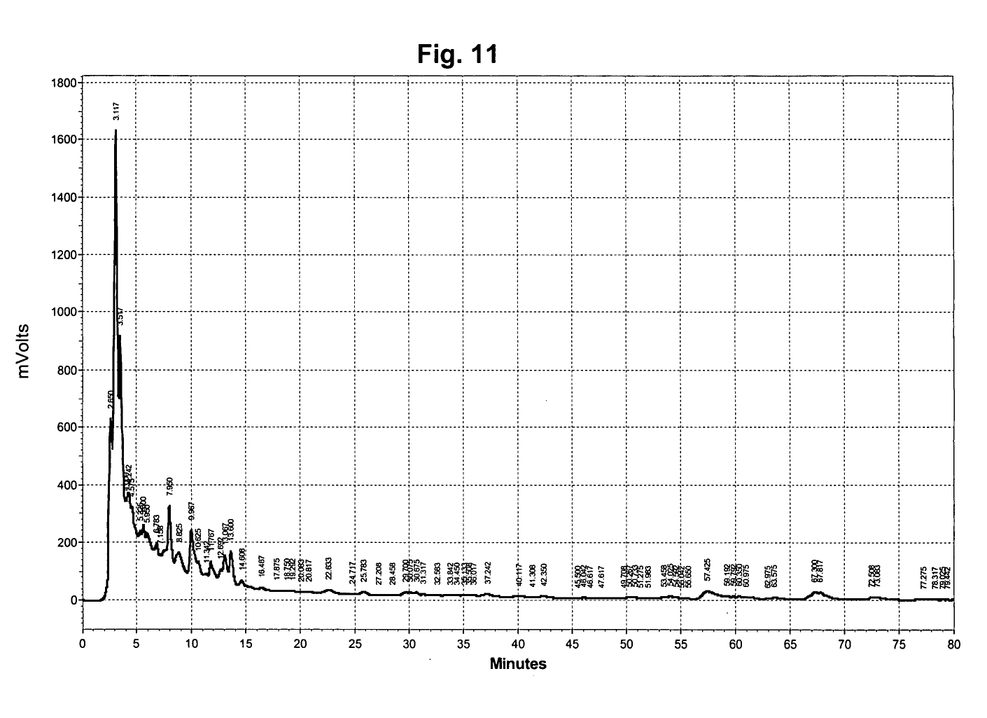

FIG. 12 depicts superimposed HPLC fingerprints of MPX, Bacopa monniera, and Centella asiatica extract.

FIG. 13 depicts comparative HPLC fingerprints of HP-200 with MPX. The highest peak for MPX was detected around 8-9 minutes with a maximum deflection of 200-250 mvolt and for HP-200 the highest was detected around 2-3 minutes with a maximum deflection of 2250-2500 mvolt.

FIG. 14 depicts a magnification of the HPLC fingerprints of MPX and HP-200 (5-30 minutes time period), indicating that MPX has a greater number of peaks and that the height of the peaks are higher as compared to HP-200.

### DETAILED DESCRIPTION

A therapeutically effective extract of Mucuna pruriens cotyledons (hereinafter referred to as “MPX”) has now been found which has been shown to be greater than or equal to raw Mucuna pruriens cotyledon powder in its antiparkinson and antioxidant activity. MPX also has the added advantages that 90% of the bulk of the preparation has been removed without loss of efficacy and that the extract can be administered both as a tablet or capsule and intraperitoneally.

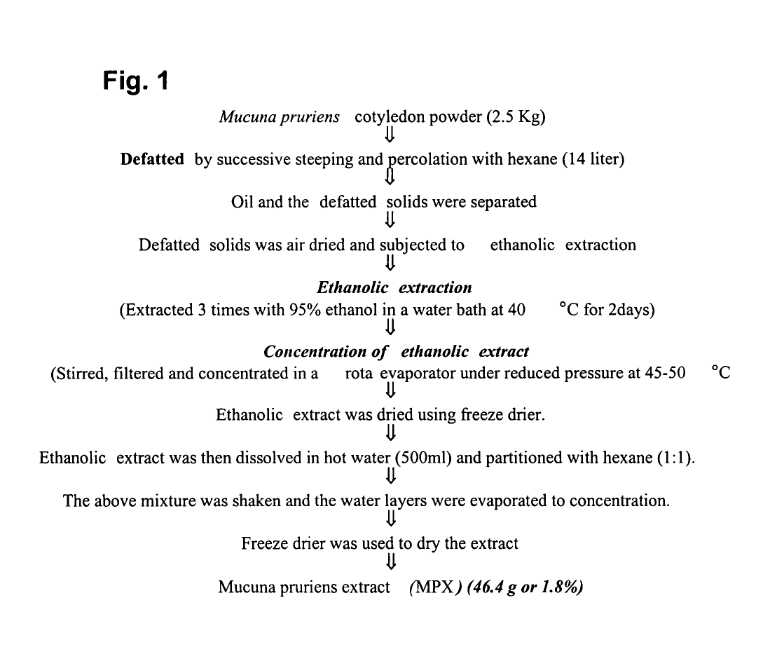

In one aspect, the present invention is a method of preparing a Mucuna pruriens cotyledon extract, wherein the bulk of an effective therapeutic dose is reduced without the loss of efficacy. FIG. 1 provides a flowchart of an exemplary extraction procedure according to the present invention, and each successive step is described in detail below. It is understood that these methods are meant to be representative, and that modification such as known in the art are contemplated as part of the present invention.

Prepare Mucuna pruriens cotyledon powder: In this step, Mucuna pruriens cotyledon powder (MPCP) is prepared for use as the starting material in the extraction process of the present invention.

To make the Mucuna pruriens cotyledon powder starting material, standard procedures for preparing a seed powder are followed. In a preferred method, harvested pods from Mucuna pruriens plants are dried in shade, pods are shelled, and the seed coat is removed manually, leaving the isolated cotyledons. The isolated cotyledons are then powdered in a mill into fine powder used as the Mucuna pruriens cotyledon powder starting material for the present invention.

Defat with Hexane: In this step, the Mucuna pruriens cotyledon powder is defatted by successive steeping and percolation with hexane, a critical step in preparing an extract that is more amenable to administration by injection. It is contemplated that other non-polar organic solvents could be used in this defatting process.

In the method of the present invention, it is preferred to defat the powder both before and after ethanol extraction as the concentration of seed oil is very high in Mucuna pruriens. The seeds contain a high concentration of non-polar seed oils. This oil fraction does not contribute to the antiparkinson and antioxidant activity of the formulation. So by removing the oils, the residue is easier to process and the active constituents are concentrated. The oils are then separated from the defatted solids by any means known in the art. For example, the oil and defatted solids are partially removed into the hexane, which is decanted from the plant residue prior to ethanol extraction. The defatted plant residue is then air dried for storage until further processing. In a preferred method, the defatted plant residue is air dried at room temperature for about 24 hours.

Extract with Aqueous Ethanol: In this step, the air dried plant residue is subjected to an ethanolic extraction process, wherein the antiparkinson/antioxidant-active portion is extracted from the plant residue into aqueous ethanol. It is contemplated that other alcohols such as methanol or n-butanol could be used in the extraction process.

As shown in FIG. 1, a preferred extraction process comprises (1) extracting the plant residue with aqueous ethanol, wherein the extraction is carried out in a water bath at about 40-50 degrees C., preferably at about 40 degrees C., with constant stirring for 2 days; (2) separating the ethanolic fraction from the remaining plant residue by any means known in the art (e.g., filtration through a filter paper) and storing the ethanolic fraction in a refrigerator until further processing; (3) repeating Steps (1) and (2) two more times; and (4) discarding the remaining plant residue. While ethanol is the preferred extracting solvent, it is contemplated that any alcohol capable of solubilizing the antiparkinson/antioxidant-active portion of the plant can be used in the present invention. In a preferred method, the air-dried plant residue is combined with aqueous ethanol, ranging from about 2% to about 95% aqueous ethanol; more preferably, from about 5% to about 50% aqueous ethanol; most preferably, from about 5% to about 20% aqueous ethanol. About 60 to 80% of the ethanol soluble material is removed in the first extraction. While most of the ethanol solubles have been removed into the aqueous ethanol fraction after three extractions, any number of extractions can be carried out without affecting the efficacy of the final product. Warming the extraction to about 40 degrees C. speeds up the extraction process; however, the extraction can be carried out at room temperature by increasing the extraction time and/or the number of extractions to optimize the removal of the ethanol solubles. Heating the extraction above 50 degrees C. should be avoided, as higher temperatures could result in the decomposition of the antiparkinson/antioxidant components in the ethanol solubles.

Concentration of Ethanolic Extract: In this step, ethanol is removed from the ethanolic fraction to give a crude extract containing the antiparkinson/antioxidant components along with some oils and water.

Once the ethanol extractions are complete, all of the ethanol fractions are combined, stirred and filtered a final time by any means known in the art (e.g, filtration through filter paper). The filtered ethanol extract is then concentrated by any means known in the art, preferably in a vacuum-equipped rota-evaporator under reduced pressure at 45-50 degrees C. Once the ethanol extractions are complete, all of the ethanol fractions are combined, stirred and filtered by any means known in the art. The filtered alcohol extract is then concentrated by removal of the ethanol by any means known in the art, preferably in a vacuum-equipped rota-evaporator under reduced pressure at 45-50 degrees C. The extract is then dried, preferably freeze dried or air dried, to remove the residual solvent.

Defat with Hexane: In this step, the crude extract is defatted again by successive steeping and percolation with hexane to remove the remaining seed oil.

The concentrated extract is suspended in hot water and partitioned with hexane (1:1) three times. The aqueous fraction with suspended solids is separated from the hexane layer, and the hexane layer containing the hexane soluble oils is discarded.

Concentration and Drying of Aqueous Fraction: In this step, the aqueous fraction with suspended solids containing the antiparkinson/antioxidant components is further concentrated and dried.

The aqueous fraction with suspended solids is brought to dryness by any means known in the art. In a preferred method, after shaking the aqueous fraction to suspend the solids, the water layer is partially removed by further concentration at 45-50 degrees C. on a vacuum-equipped rota-evaporator at reduced pressure, optionally adding some absolute ethanol to speed the concentration process. The resulting concentrated extract is taken to dryness using a freeze drier according to manufacturer's instructions, resulting in the solid MPX extract of the present invention. The solid MPX extract is preferably stored in a brown bottle at refrigerator temperature to protect against decomposition by heat and light, thus providing a shelf life of at least six to eight months.

As indicated in FIG. 1, in a representative extraction process, about 46 grams of MPX was recovered from 2.5 kg crude Mucuna pruriens cotyledon powder (1.8% yield). It is to be understood that as a natural product, the Mucuna pruriens cotyledons will vary in their exact chemical composition, so that the yield of MPX can vary (about 1.5% to 2.1%). To maximize shelf life stability, MPX is preferably stored in dry powder form until just before administration, thus avoiding problems related with storage of plant based products and accelerated degradation of active ingredients in solution.

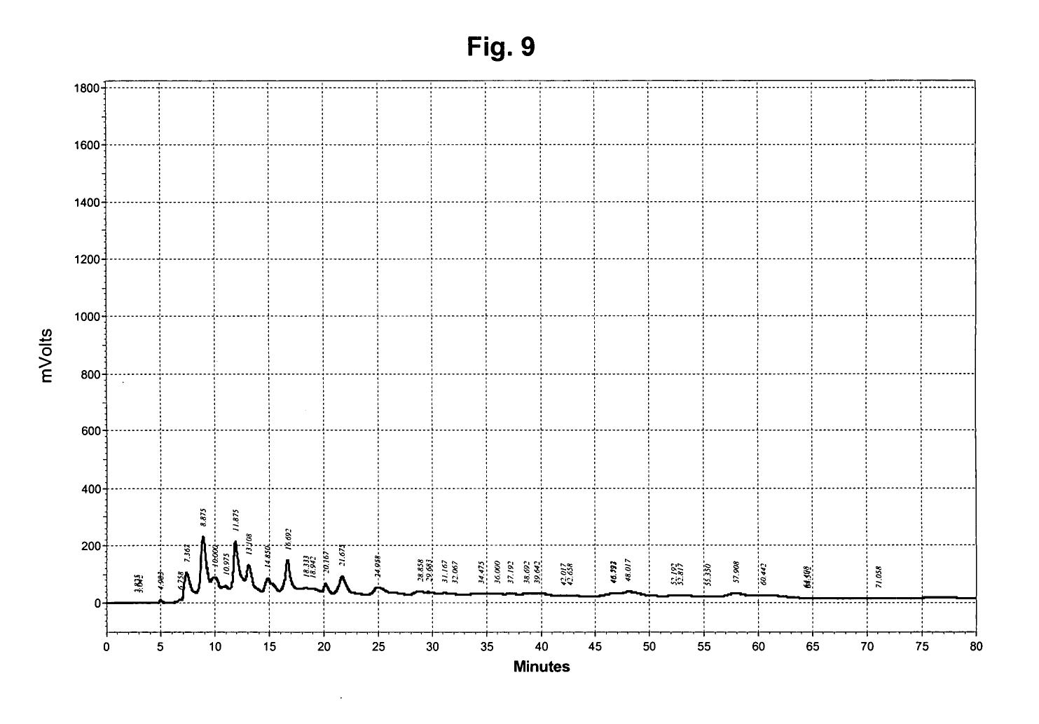

As an extract of a natural product, the chemical composition of MPX is only partially understood. In FIG. 9, a fingerprint HPLC is given of at least 39 peaks, which indicates the presence of 39 different fat soluble components. Certain water and fat soluble chemical components have been characterized and identified, and Table I gives a comparison of the amount of identified chemical components (per mg) of the Mucuna pruriens cotyledon extract of the present invention (MPX) to the amount found in the Mucuna pruriens cotyledon powder (MPCP), i.e., the starting material from which the extract was made.

TABLE I
Comparison of the Relative Amount of
Known Components in MPX and MPCP

| COMPONENT | MPX | | MPCP | |
|---|---|---|---|---|
| NADH | 2.59 ± 0.32 | μg | 1.41 ± 0.14 | μg |
| Polyphenols | 35.81 ± 0.01 | μg | 82.76 ± 3.65 | μg |
| Sulfhydryl Content | 100.69 ± 13.25 | μg | 30.74 ± 5.99 | μg |
| Coenzyme Q-10 | 13.39 ± 1.37 | ng | 2.32 ± 0.33 | ng |
| Levodopa | 51.55 ± 3.58 | μg | 39.21 ± 4.22 | μg |
| Proteins | 79.11 ± 0.02 | μg | 9.5 ± 0.6 | μg |

MPX contains levodopa; however, the diminutive amount of levodopa reported in Mucuna pruriens seeds is unlikely to account for improvement of symptoms of Parkinson's disease alone (Manyam B V. 1990. “Paralysis agitans and levodopa in “Ayurveda,” ancient Indian medical treatise. Movement Disorders 5:47-48; Hussain G and Manyam B V. 1997. “Mucuna pruriens proves more effective than L-DOPA in Parkinson's disease animal model,” Phytotherapy Res 11:419-423 (each of these references are hereby incorporated by reference in their entirety)). As indicated above, MPX also contains several components that contribute to its antiparkinson and/or antioxidant activity.

The current existing rational management of Parkinson's disease requires treatment with levodopa in combination with: (1) dietary revision, especially to lower calories; (2) rebalancing of essential fatty acid intake away from pro-inflammatory and toward anti-inflammatory prostaglandin; (3) aggressive repletion of glutathione and other nutrient antioxidants and cofactors; (4) energy nutrients acetyl L-camitine, coenzyme Q-10, NADH, and the membrane phospholipid phosphatidylserine; and (5) chelation as necessary for heavy metals. Thus, neuroprotection rendered by the MPX extract of the present invention is due to both cumulative and synergistic effects of components present.

MPX has a significant amount of nicotinamide adenine dinucleotide (NADH), a coenzyme whose primary role is in the electron transport chain to synthesize the high energy molecule ATP and reported to be effective in helping Parkinson's patients by boosting endogenous dopamine production. The presence of polyphenols in MPX contributes to MPX's significant antioxidant, chelating, and the lipid peroxidation inhibitory properties (Examples 2-7). In keeping with is antioxidant and lipid peroxidation inhibitory properties, MPX has significant amount of sulthydryl groups, which can undergo redox reactions to scavenge hydroxyl radicals. Considering the decreased mitochondrial complex-I activity in the substantia nigra of patients with Parkinson's disease, the ability of thiol containing MPX to significantly increase in brain complex-I activity (Example 8) contributes to its efficacy in treating Parkinson's disease. MPX contains natural coenzyme Q-10, thus contributing to its efficacy in the treatment of Parkinson's disease, and the amount of coenzyme Q-10 in MPX is higher than the amount found in the raw Mucuna pruriens cotyledon powder (MPCP).

In another aspect of the present invention, MPX is suitable as a therapeutic for treatment of Parkinson's disease and related disorders. Therapeutic compositions comprising MPX may be employed in any conventional manner for the treatment of Parkinson's disease or related disorders. Such methods of treatment, their dosage levels and requirements would be understood by one of ordinary skill in the art from available methods and techniques. For example, a therapeutic composition useful in this invention comprises a therapeutically effective amount of MPX combined with a pharmaceutically acceptable carrier for administration to a patient suffering from Parkinson's disease or a related disorder. The therapeutic compositions comprising MPX useful in the present invention can also be co-administered either concomitantly or sequentially with other therapeutic agents known to be effective in the treatment of Parkinson's disease or a related disorder.

The therapeutic efficacy of MPX as an antiparkinson drug is demonstrated in in vitro studies given in Examples 2-7 and an in vivo study given in Example 8. In Example 2, the antiparkinson activity of MPX is demonstrated in terms of dose-dependent contralateral rotation in 6-OHDA treated rats following intraperitoneal administration of MPX. In vitro antioxidant effects of MPX is demonstrated in detail with respect to various free radicals: (1) diphenylpicrylhydrazyl (DPPH) radicals in Example 3; (2) 2,2′-azinobis-(3-ethylbenzothiazoline-6-sulfonate in Example 4; (3) 2,3- and 2,5-dihydroxybenzoic acid (DHBA) in Example 5; (4) reactive oxygen species from hydrogen peroxide induced lipid peroxidation in Example 6; and (5) cupric radicals provided in a commercially available total antioxidant activity kit in Example 7. Importantly, MPX was shown to increase mitochondrial complex-I activity in vivo (Example 8). In summary, MPX has been shown to have significant antioxidant activity, thus providing a means to control both the symptomatic motor abnormalities associated with Parkinson's disease and to delay the progression of the disease in patients with Parkinson's disease without the adverse effect of dyskinesia associated with long-term levodopa therapy. In keeping with recent research trends showing the role of free radical oxidation in the etiology of Parkinson's disease, MPX provides a means of protecting neurons by blocking oxidative stress induced neurodegeneration.

For the treatment of Parkinson's disease or related disorders, the therapeutic compositions useful in the present invention comprise an amount of MPX to provide between 8.6 mg MPX/kg body weight per day in divided doses to about 10.25 mg MPX/kg body weight per day in divided doses upon oral administration. Under conditions where oral administration may not be feasible, such as pre-surgical or immediate post-surgery conditions, MPX can be administered parenterally at between 1.7 mg MPX/kg body weight per dose to about 2.05 mg MPX/kg body weight per dose. In accordance with the present invention, it is contemplated that MPX can be administered alone or in combination with other drugs as part of a treatment regimen for Parkinson's disease or related disorders.

Therapeutic compositions comprising MPX useful in the present invention can be administered with any suitable pharmaceutically acceptable carrier. Pharmaceutically acceptable carriers are well known in the art and are disclosed, for instance, in Sprowl's American Pharmacy, Dittert, L. (ed.), J. B. Lippincott Co., Philadelphia, 1974, and Remington's Pharmaceutical Sciences, Gennaro, A. (ed.), Mack Publishing Co., Easton, Pa., 1985 (each of these references are hereby incorporated by reference in their entirety).

Therapeutic compositions comprising MPX useful in the treatment of Parkinson's disease and related disorders can be formulated as parenteral (injectable) solutions or lyophilized powders for parenteral administration. Powders may be reconstituted by addition of a suitable diluent or other pharmaceutically acceptable carrier prior to use. The liquid formulation is generally a buffered, isotonic, aqueous solution, but a lipophilic carrier, such as propylene glycol optionally with an alcohol, can be used. Examples of suitable diluents are normal isotonic saline solution, standard 5% dextrose in water of buffered sodium or ammonium acetate solution. Such a formulation is especially suitable for parenteral administration, but can also be used for oral administration or contained in a metered dose inhaler of nebulizer for insufflation or spray or drops to the nasal mucosa. It may be desirable to add excipients such as ethanol, polyvinylpyrrolidone, gelatin, hydroxy cellulose, acacia, polyethylene glycol, mannitol, sodium chloride or sodium citrate. Other administration routes include but are not limited to oral, inhalation spray, topical, rectal, nasal, buccal, vaginal or implanted reservoir methods.

In a preferred embodiment, MPX powder is dispersed just prior to administration into a suitable sterile pharmaceutical carrier such as water and dimethyl sulfoxide. For example, freeze dried and finely powdered MPX (50 mg/ml) is dispersed in sterile water containing benzalkonium chloride (0.04%) and sodium benzoate (0.01%) (The chemical nature of MPX and its acidic pH 5.2 render it unsuitable for growth of microbes; however, use of a combination of antibacterial and antifungal substances that has been officially listed in the United States Pharmacopoeia is recommended.)

Alternately, the therapeutic compositions comprising MPX useful in the present invention may be encapsulated, tableted or prepared in an emulsion or syrup for oral administration. Pharmaceutically acceptable solid or liquid carriers may be added to enhance or stabilize the preparations, or to facilitate preparation. Liquid carriers include syrup, soybean oil, peanut oil, olive oil, glycerin, saline, ethanol, and water. Solubilizing agents, such as dimethyl sulfoxide, ethanol or formamide, may also be added. Carriers, such as oils, optionally with solubilizing excipients, are especially suitable. Oils include any natural or synthetic non-ionic water-immiscible liquid, or low melting solid capable of dissolving lipophilic compounds. Natural oils, such as triglycerides are representative.

Solid carriers include starch, lactose, calcium sulfate dihydrate, terra alba, magnesium stearate or stearic acid, talc, pectin, acacia, agar or gelatin. Solubilizing agents, such as dimethyl sulfoxide or formamide, may also be added. The carrier may also include a sustained release material such as glyceryl monostearate or glyceryl distearate, alone or with a wax. The therapeutic compositions can be made in solid form following the conventional techniques of pharmacy involving milling, mixing, granulating, and compressing for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation can be administered directly p.o. or filled into a soft gelatin capsule. For rectal administration, a pulverized powder of the therapeutic agents useful in the present invention may be combined with excipients such as cocoa butter, glycerin, gelatin or polyethylene glycols and molded into a suppository.

Based on the antiparkinson and antioxidant activity of MPX from Mucuna pruriens cotyledons, it is contemplated that extractions of cotyledons from other Mucuna species may also yield effective therapeutics for Parkinson's disease and related disorders and are considered to fall within the scope of the invention.

### EXAMPLE 1

### MPX Extraction Process

As illustrated in FIG. 1, MPX was prepared from crude Mucuna pruriens cotyledons according to the following method. Raw Mucuna pruriens cotyledons (2.5 kilograms) was ground to a powder using a standard pharmaceutical mesh for powder. The resulting powder is referred to herein as Mucuna pruriens cotyledon powder (MPCP).

The ground powder (2.5 kg) is first defatted by successive steeping and percolation with hexane (14 liters). The oils and defatted solids were separated by removing the hexane layer, and the oil fraction was discarded.

The defatted solids were then extracted three times in 95% ethanol in a water bath at 40 degrees C. with constant stirring for 2 days: Extraction 1:5 liters ethanol for 2 days; Extraction 2:5 liters ethanol for 2 days; and Extraction 3:3.5 liters ethanol for 2 days. At the conclusion of each extraction, the extraction fluid was separated from the solid portion by gravity filtration through a Whatman filter paper, and the ethanolic fraction was stored at refrigerator temperature. At the conclusion of the extraction process, the three separate ethanolic fractions were combined, stirred, and then filtered by gravity filtration through a Whatman filter paper. The combined filtrate (13.5 liters) was concentrated in a rota-evaporator under reduced pressure at 45-50 degrees C. The concentrated ethanolic extract (220 g) was suspended in hot water (500 ml) and partitioned with hexane (1:1). The hexane layer was removed and discarded. The remaining aqueous mixture was shaken, and the water layer was evaporated to concentration in a rota-evaporator under reduced pressure at 45-50 degrees C., using ethanol to help azeotrope the water. The concentrated extract (approximately 80 g) was dried in a freeze drier to yield 46.4 g MPX (1.8%).

### EXAMPLE 2

### Antiparkinson Motor Activity of MPX at Various Concentrations

The antiparkinson activity of MPX was demonstrated as dose-dependent contralateral rotations in 6-OHDA treated rats, a valid rat model for Parkinson's disease. Further, the effectiveness of intraperitoneal administration of MPX was confirmed.

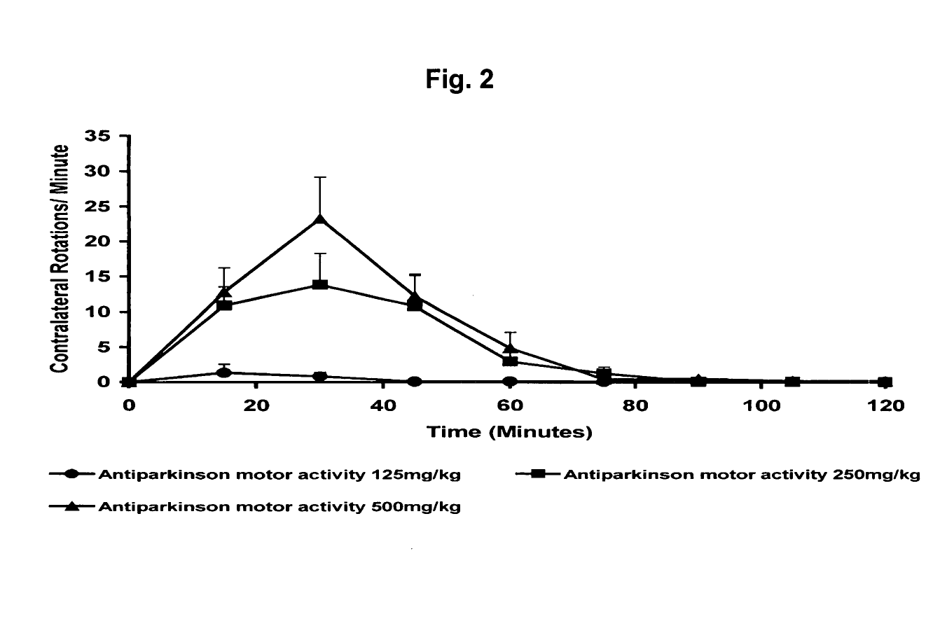

Rats were injected with 6-OHDA in the right striatum using a stereotaxic frame. Following validation of the intrastriatal 6-OHDA injection with amphetamine, the animals were treated with MPX at a dose of 125 milligram/kilogram body weight intraperitoneally (n=6), 250 milligram/kilogram body weight intraperitoneally (n=6), or 500 milligram/kilogram body weight intraperitoneally (n=6). Controls received no drug. Contralateral rotation (opposite to the side of 6-OHDA lesion) was recorded over time for 120 minutes, as a measure of antiparkinson activity using a rotometer. As shown in FIG. 2, the results indicated that MPX at all three doses caused dose-dependent contralateral rotation in 6-OHDA treated rats, indicating its antiparkinson activity. The maximum activity was observed at 30 minutes post-administration of MPX.

### EXAMPLE 3

### Antioxidant Effect of MPX on Diphenylpicrylhydrazyl (DPPH) Radicals

This experiment was performed using standard spectrophotometric techniques. To demonstrate the in vitro antioxidant effect of MPX on DPPH radicals, a colorimetric assay was performed by adding 1:10 μg MPX (10 mg MPX in 1 ml water) to 200 μl DPPH solution (2 mg DPPH in 100 ml methanol) in a 96 well microplate. The absorbance at 517 m was then measured using a Bio-Tek Synergy-HT spectrophotometric plate reader (Winooski, Vt.) after 3 minutes. Methanol was used as the blank solution.

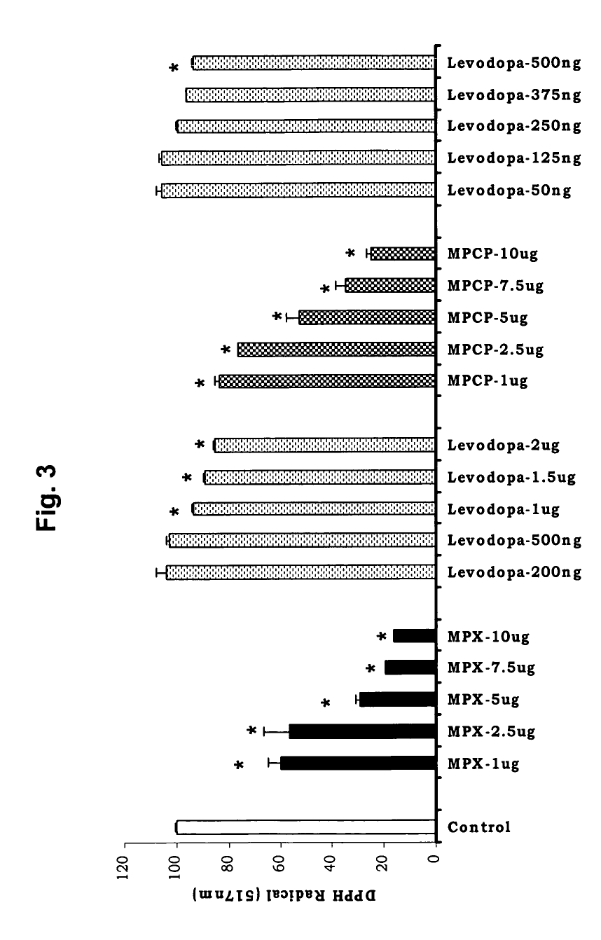

MPX exhibited dose-dependent DPPH radical scavenging activity (FIG. 3). MPX exhibited dose-dependent and significant DPPH radical scavenging activity. At a concentration of 100 μg/ml in water, MPX scavenged 89% of the DPPH radical. MPX also had higher antioxidant activity as compared to Mucuna pruriens cotyledon powder (MPCP).

### EXAMPLE 4

### Antioxidant Effect of MPX on ABTS Radicals

This experiment was performed using standard spectrophotometric techniques. The 2,2′-azinobis-(3-ethylbenzothiazoline-6-sulfonate) (ABTS) radical decoloration method is capable of determining antioxidant properties in natural compounds. ABTS (1.7 mg in 1 ml water) was incubated with peroxidase (metmyoglobin; 1 mg in 1 ml water) and hydrogen peroxide (0.3%). This resulted in production of blue green colored radical cation ABTS. The intensity of the color was detected using a spectrophotometer at 734 nm. To the ABTS radical, MPX (10 mg in 1 ml water) was added, and the reduction in intensity of the blue-green color was detected using the same spectrophotometric setting at 734 nm. The difference of the initial blue-green color intensity minus the reduction of color intensity by MPX is an indication of the antioxidant activity of MPX. The procedure was repeated at different doses of MPX.

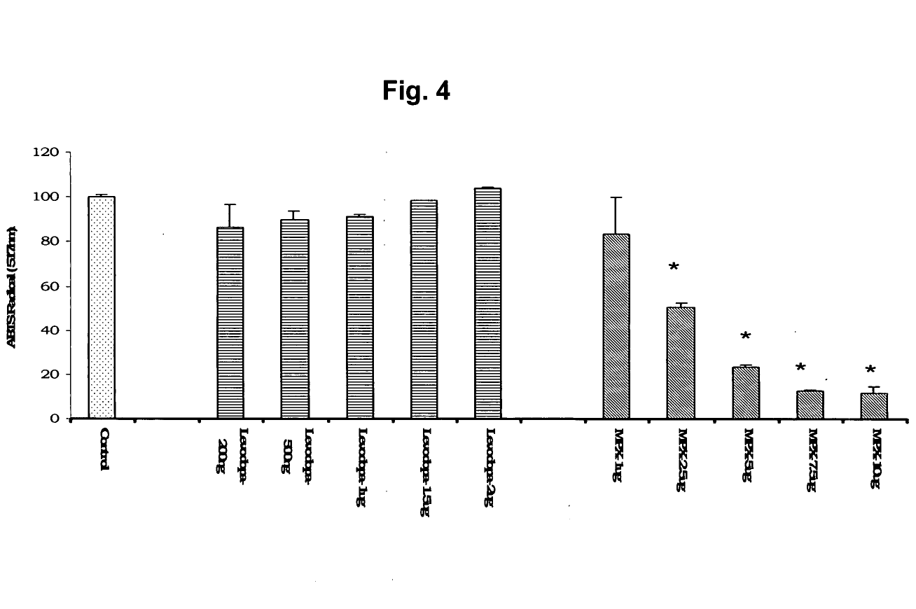

MPX dose-dependently scavenged the ABTS radical starting at 25 μg/ml, reaching 95% at 100 μg/ml (p<0.001) antioxidant activity (FIG. 4).

### EXAMPLE 5

### Antioxidant Effect of MPX on Hydroxyl Radicals

This experiment was performed using standard HPLC-electrochemical detection technique. Hydroxyl radicals were generated by the reaction of ferrous ammonium sulfate (10 mM) and citric acid (10 mM) in the Fenton Reaction and then trapped by using salicylic acid (10 mM) to form hydroxyl radical adducts 2,3- and 2,5-dihydroxy benzoic acid (DHBA). DHBA was measured at pico mole concentration using HPLC-electrochemical detection with a C-18 reverse phase column and an acetonitrile aqueous mobile phase.

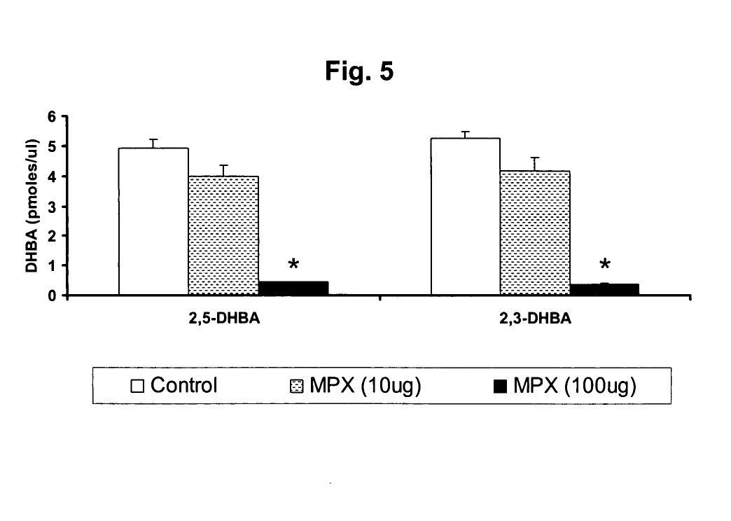

MPX (10 mg in 1 ml water) significantly (p<0.001) exhibited antioxidant activity by dose-dependently scavenging the hydroxyl radicals generated by the Fenton reaction (FIG. 5).

### EXAMPLE 6

### Antioxidant Effect of MPX on Hydrogen Peroxide Induced Lipid Peroxidation

This experiment was performed using standard spectrophotometric techniques. Hydrogen peroxide-induced lipid peroxidation is a model commonly used to examine the antioxidant effect of different drugs. Control rat brain homogenate (1 mg in 10 μl PBS) was incubated with hydrogen peroxide (10 μM) to result in excessive formation of lipid peroxides. Thiobarbituric acid (TBA) (5 mg in 1 ml PBS) was then added to the lipid peroxides, and this resulted in the development of pink color due to the formation of thiobarbituric acid reactive substances (TBARS), which was measured spectrophotometrically at 532 nm.

Several doses of MPX (10 mg in 1 ml water) were incubated with rat brain homogenate and hydrogen peroxide (10 μM) and then incubated with TBA to detect the lipid peroxides formed. The difference between the control and the MPX treated sample is the measurement of decrease in TBARS formation, reflecting inhibition of lipid peroxidation, which in turn is the measurement of antioxidant activity.

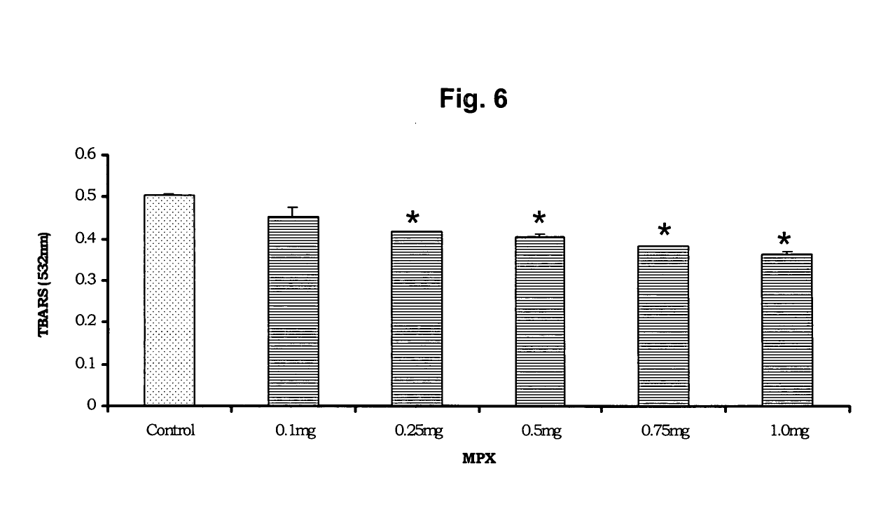

MPX caused a significant antioxidant activity in a dose-dependent manner (FIG. 6).

### EXAMPLE 7

### Total Antioxidant Effect of MPX

This experiment was performed using standard spectrophotometric techniques. The total antioxidant effect of MPX was assessed using a commercially available kit (BIOXYTECH AOP-490™ purchased from OXIS Health Products, Inc. Portland, Oreg.). The antioxidant assay was based upon the reduction of divalent metal Cu⁺⁺ to monovalent Cu⁺. Bathocuproine (chromogenic reagent, 2,9-dimethyl-4,7-diphenyl-1,10-phenanthroline), was added to the monovalent Cu⁺ which selectively forms a 2:1 complex that was detected at 490 nm using a spectrophotometer. Uric acid, a well established antioxidant, was used as a known internal standard. Various concentrations of MPX ranging from 1-10 μg (10 mg in 1 ml water) were incubated with the Cu⁺⁺ reagents supplied in the kit. Bathocuproine was added to this reaction, and the reading was measured at 490 nm.

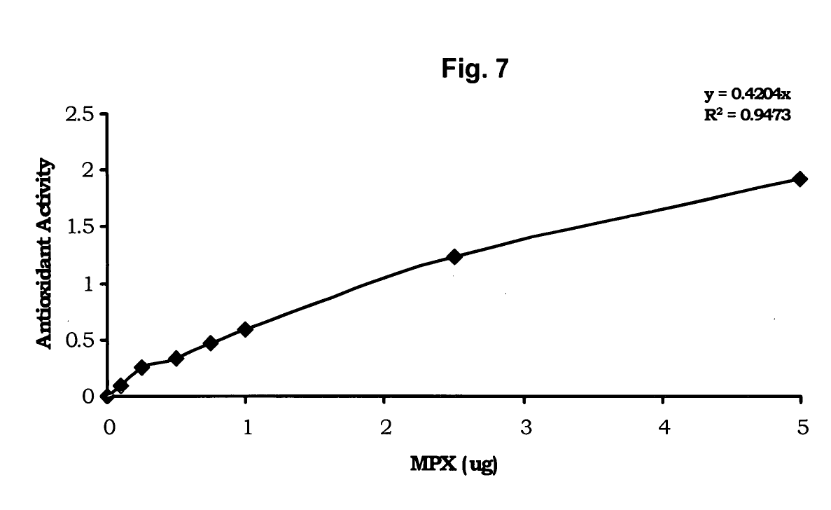

MPX exhibited dose-dependent increase in the bathocuproine complex formation as indicated by the increase in absorbance (FIG. 7). The results were expressed as the antioxidant activity equivalent to uric acid. Data represents mean±SEM (n=6). The significant chelating ability and antioxidant activity of MPX is its ability to provide neuroprotection to the dopaminergic neurons in Parkinson's disease.

### EXAMPLE 8

### In Vivo Effect of MPX on Mitochondrial Complex-I Activity

Various doses of MPX were administered intraperitoneally to Sprague Dawley rats for 2 weeks, while a control group was treated with saline. The rats were sacrificed one hour after the last injection, and the mitochondrial (P₂) fraction was prepared from the dissected rat brains. Mitochondrial complex-I activity was based on the oxidation of NADH (5.6 mg in 1 ml water) measured at 340 nm (Ramsay et al. 1986. “Inhibition of mitochondrial NADH dehydrogenase by pyridine derivatives and its possible relation to experimental and idiopathic parkinsonism,” Biochem Biophys Res Commun 135:269-275 (this references is hereby incorporated by reference in its entirety)).

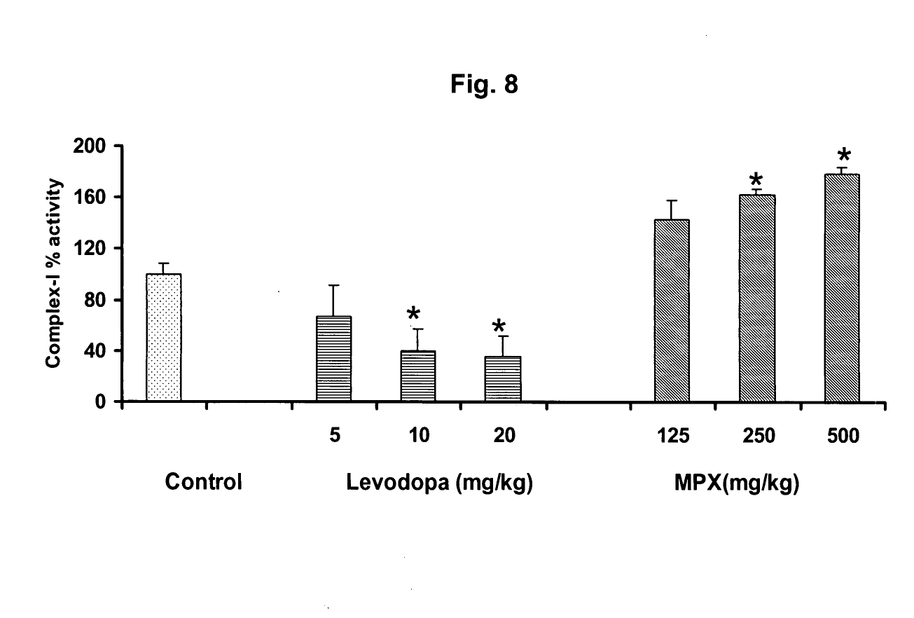

MPX dose-dependently increased the complex-I activity (FIG. 8). Because complex-I activity is known to decrease in Parkinson's disease, the ability of MPX to reverse the complex-I deficit by increasing complex-I activity is an important benefit. On the contrary, synthetic Levodopa (equivalent does and treatment) showed significantly inhibitory effect on complex-I.

### EXAMPLE 9

### Chemical Analysis of MPX

A reference standard of MPX was injected into HPLC to obtain a fingerprint. The mobile phase consisted of methanol and hexane, and the peaks were detected using ultraviolet detector. MPX was homogenized in ethanol (10% w/v) and mixed with hexane (2:5-ethanol: hexane v/v). The above mixture was shaken for 10 minutes in the dark. The hexane layer was removed by centrifugation, evaporated using Meyer N-EVAP analytical evaporator, and then redissolved in mobile phase (methanol 75% and hexane 25%). C-18 Hypersil-ODS silica based column was used in our studies. Hypersil ODS phases are based on completely porous spherical silica particles with a pore size of 120 D. The column length was 125 mm, and the diameter was 3 mm. The flow rate was 0.3 ml/min, and the UV-detection was performed at 225 nm.

A fingerprint of MPX is given in FIG. 9. About 39 peaks were noted in the chromatogram, indicating the presence of at least 39 unknown compounds in MPX.

### EXAMPLE 10

### Comparative HPLC Analysis of Mucuna pruriens MPX, Bacopa monniera and Centella asiatica

A comparative HPLC fingerprint analysis was performed using extracts of MPX, Bacopa monniera and Centella asiatica.

Each extract of Mucuna pruriens (MPX), Bacopa monniera and Centella asiatica (25mg) was weighed accurately. Ethanol (250 microliters) was then added to the extract and sonicated. Hexane (625 microliters) was then added to the sonicated mixture. The mixture was shaken vigorously for 15 minutes in the dark. The hexane layer was removed and evaporated at room temperature. The concentrate was then resuspended in 100 microliters mobile phase (methanol-75: hexane-25) and injected in HPLC connected with an ultraviolet detector. The peaks were detected at 225 nm with a flow rate of 0.3 milliters/minute, and the duration of the HPLC run for the peak detection was 80 minutes. Shimadzu LC-IOAD HPLC with UV detector SPD-10AV was used for this experimental procedure.

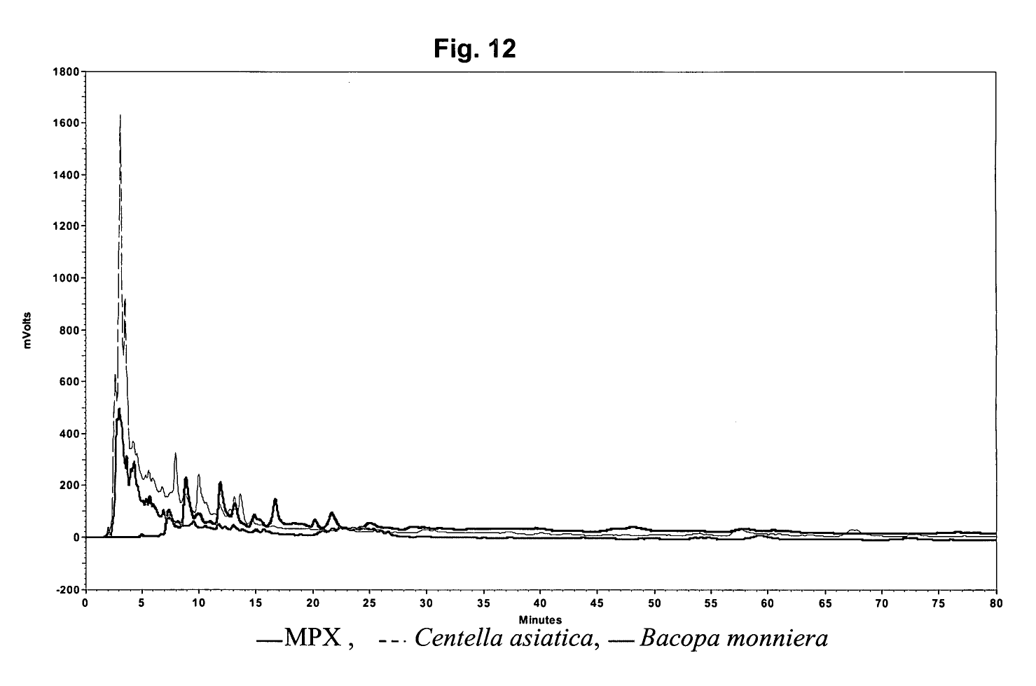

The resulting HPLC fingerprints for the three extracts are given FIG. 9, 10 and 11. There were 39 peaks detected in the extract of Mucuna pruriens (MPX) compared to 65 and 75 peaks detected in the Bacopa monniera and Centella asiatica extracts, respectively. In the HPLC fingerprint, the Mucuna pruriens extract's highest peak was detected around 8.8 minutes with a maximum deflection of 200-250 mvolt. In Bacopa monniera, the highest peak was detected around 2.8 minutes with a maximum deflection of 400-500 mvolt. In Centella asiatica, the highest peak was detected around 3.1 minutes with a maximum deflection of 1600-1700 mvolt. FIG. 12 depicts superimposed HPLC fingerprints of MPX, Bacopa monniera, and Centella asiatica extract. As shown in FIG. 12, in spite of same amount of the extracts administered into HPLC, MPX's pattern and number of peaks are different as compared to the peaks obtained from Bacopa monniera and Centella asiatica.

### EXAMPLE 11

### Comparative HPLC Analysis of Mucuna pruriens MPX and HP200

A comparative HPLC fingerprint analysis was performed using HP-200 and MPX. HP-200 is a commercially available Mucuna pruriens powder, which is manufactured by Zandu Pharmaceuticals, Bombay (currently Mumbai) India. MPX was prepared according to Example 1. MPX is an extract as compared to HP-200, which contains the raw Mucuna pruriens cotyledon powder as its active ingredient.

HP-200 (25 milligrams) was weighed accurately. Ethanol (250 microliters) was then added to the extract and sonicated. Hexane (625 microliters) was then added to the sonicated mixture. The mixture was shaken vigorously for 15 minutes in the dark. The hexane layer was removed and evaporated at room temperature. The concentrate was then resuspended in 100 microliters mobile phase (methanol-75:hexane-25) and injected in HPLC connected with an ultraviolet detector. The peaks were detected at 225 nm with a flow rate of 0.3 milliliters/minute, and the duration of the HPLC run for the peak detection was 80 minutes. Shimadzu LC-10AD HPLC with UV detector SPD-10AV was used for this experimental procedure.

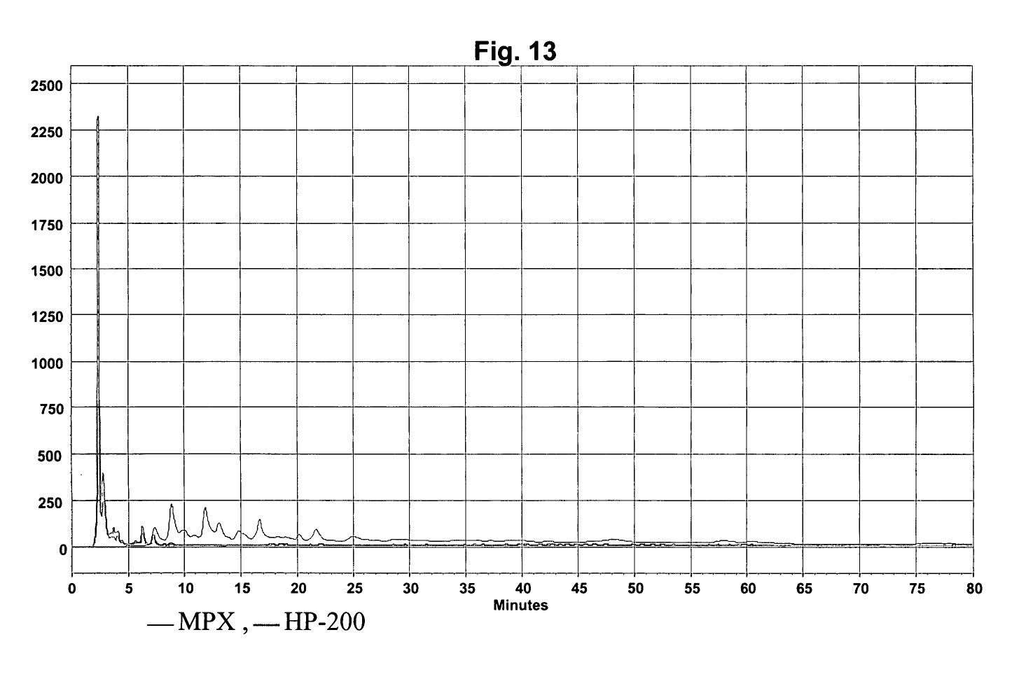

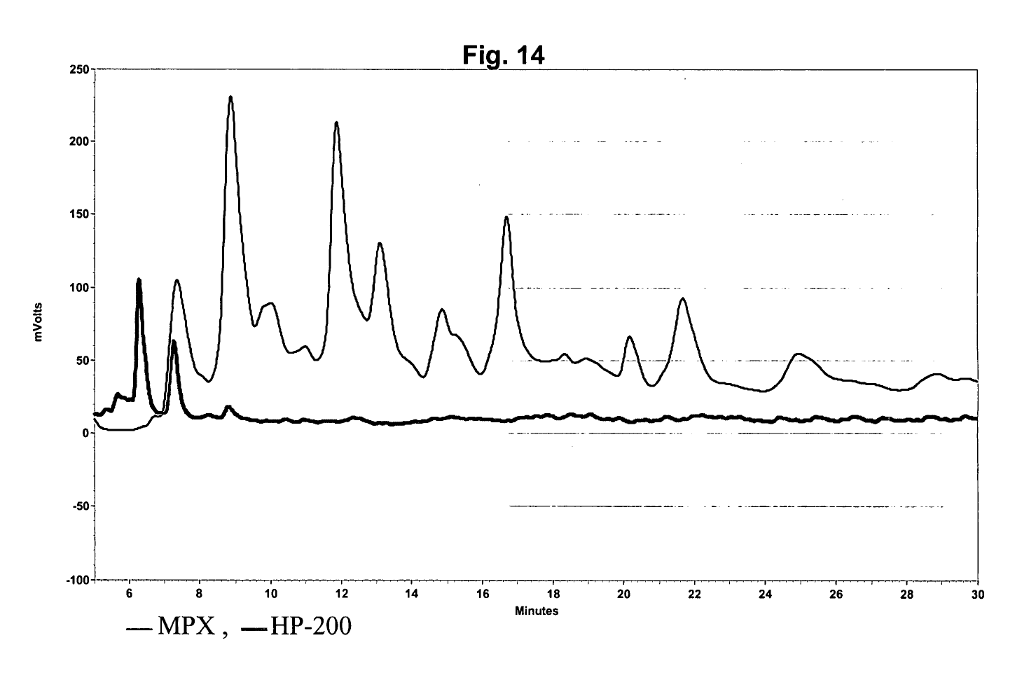

The results of the HPLC fingerprint analysis are given in FIG. 13, which depicts comparative HPLC fingerprints of HP-200 with MPX. HP-200's highest peak was detected around 2-3 minutes with a maximum deflection of 2250-2500 mvolt. However, MPX had a highest peak around 8-9 minutes with a maximum deflection of 200-250 mvolt. Magnification of the fingerprint region between the 5 minute and 30 minute time period is given in FIG. 14. The chromatogram clearly shows that MPX has a greater number of peaks than HP-200, reflecting the presence of a greater number of compounds in MPX, and that the height of the peaks in MPX are higher as compared to HP-200. Thus, in spite of same amount by weight of the HP-200 and MPX injected into the HPLC, MPX's HPLC fingerprint pattern is different from HP-200, indicating the uniqueness of the MPX extract with different chemical components present in them.

1. A method for preparing an extract of Mucuna pruriens cotyledon powder comprising
defatting Mucuna pruriens cotyledon powder to form defatted solids;
extracting the defatted solids with aqueous ethanol;
separating the aqueous ethanol from the defatted solids to form an aqueous ethanolic extract;
defatting the aqueous ethanolic extract;
removing the ethanol from the aqueous ethanolic extract to form an aqueous extract;
and optionally, removing the water from the aqueous extract to form a purified Mucuna pruriens extract powder.

2. A purified Mucuna pruriens extract powder made according to the extraction method of claim 1.

3. A therapeutic composition comprising purified Mucuna pruriens extract powder made according to the extraction method of claim 1.

4. A method for treating Parkinson's disease or a related disorder comprising administering an effective therapeutic amount of the purified Mucuna pruriens extract powder made according to the extraction method of claim 1.

5. A therapeutic composition comprising purified Mucuna pruriens extract having the chemical fingerprint shown in FIG. 9.

6. A method for preparing an extract from a Mucuna species comprising:
contacting a Mucuna species with a non-polar organic solvent; and
contacting the Mucuna species with an alcohol reagent to form an extract.

7. The method of claim 6, wherein the Mucuna species is selected form the group consisting of Mucuna pruriens, Mucuna andrena, Mucuna birdwoodiana, Mucuna deeringiana, Mucuna holtini, Mucuna irukande, Mucuna mutisiana, Mucuna sempervirens and Mucuna slonei.

8. The method of claim 6, wherein the non-polar organic solvent comprises hexane.

9. The method of claim 6, wherein the alcohol reagent is ethanol.

10. The method of claim 6, further comprising separating the solvent and the alcohol reagent from the Mucuna species to form a Mucuna species extract.

11. The method of claim 6, wherein the Mucuna species is contacted with the non-polar organic solvent before and after contact of the Mucuna species with the alcohol reagent, and wherein the solvent and the alcohol reagent are each thereafter separated from the Mucuna species to form the Mucuna species extract.

12. The method of claim 6, wherein the Mucuna species comprises Mucuna pruriens cotyledon powder.

13. The method of claim 6, wherein the Mucuna species comprises a powdered cotyledon of the Mucuna species.

14. A therapeutic composition comprising a Mucuna species extract prepared by the method of claim 6.

15. The therapeutic composition of claim 14, further comprising a pharmaceutically acceptable carrier, excipient, diluent or combination thereof.

16. A Mucuna pruriens extract (MPX) comprising the components in the concentrations listed in Table I for MPX.

17. A composition comprising an amount of an Mucuna pruriens extract (MPX) having an in vitro or in vivo antiparkinson or antioxidant effect equal to or greater than an amount of an Mucuna pruriens cotyledon powder (MPCP), wherein the amount of the MPX is about 90% less than the amount of the MPCP.

18. A method for treating or managing Parkinson's disease or a related disorder comprising administering to a subject in need of such treatment or management, an effective amount of the Mucuna species extract of claim 5.

19. A method for treating or managing Parkinson's disease or a related disorder comprising administering to a subject in need of such treatment or management, an effective amount of the Mucuna species extract prepared by the method of claim 6.

20. A method for treating or managing Parkinson's disease or a related disorder comprising administering to a subject in need of such treatment or management, an effective amount of the MPX of claim 17.
